(19) United States
(12) Patent Application Publication
(10) Pub. No.: US 2026/0115129 A1
(43) Pub. Date: Apr. 30, 2026

(54) METHOD FOR INCREASING EFFECTIVE POLYPHENOL COMPONENTS USING HIGH-TEMPERATURE AND PRESSURIZATION STEAM EXTRACTION METHOD

(71) Applicant: SUMSUMBIOCO., LTD, Jeollanam-do (KR)

(72) Inventors: Moon Hee Choi, Gwangju (KR); Hyun Jae Shin, Gwangju (KR)

(21) Appl. No.: 19/373,409

(22) Filed: Oct. 29, 2025

(30) Foreign Application Priority Data
Oct. 30, 2024 (KR) 10-2024-0150611

(51) Int. Cl. A61K 8/9789; A61K 8/14; A61K 8/36; A61K 8/9728; A61Q 19/08

(52) U.S. Cl. CPC A61K 8/9789; A61K 8/14; A61K 8/361; A61K 8/9728; A61Q 19/08

(57) ABSTRACT

The present disclosure relates to an extraction method capable of increasing effective components such as polyphenols and flavonoids using a high-temperature and pressurization steam extraction process. The high-temperature and pressurization steam extraction process of the present disclosure can significantly increase the extractive contents of protocatechuic acid and chlorogenic acid, as well as gallic acid, which is a powerful antioxidant as an effective component. In addition, the extractive contents of catechin and tannic acid, which are effective in inhibiting wrinkle formation, can also be effectively increased. The extract extracted through this extraction method contains a high content of polyphenol and flavonoid components, and therefore, the composition of the present disclosure containing the extract as an effective component can be usefully used as a variety of functional cosmetic compositions such as antioxidant, wrinkle improvement, and whitening components.

### BACKGROUND

The present disclosure relates to an extraction method capable of increasing effective components such as polyphenols and flavonoids using a high-temperature and pressurization steam extraction process.

The skin, in direct contact with the external environment, functions to protect against temperature, humidity, antigens, and ultraviolet (UV) rays. However, physical and chemical stimuli such as external pollutants, UV rays, and stress can deteriorate skin functions. As we age, cell proliferation and immune activity weaken, preventing skin cells from quickly repairing damage and leading to an overall deterioration of skin composition, resulting in wrinkles, loss of elasticity, and keratinization.

Skin aging is broadly divided into intrinsic and extrinsic aging. Intrinsic aging is a natural phenomenon that is clinically characterized by decreased elasticity, rough skin texture, deep wrinkles, and pigmentation. Extrinsic aging is caused by external environmental factors such as UV rays, reactive oxygen species (ROS), and stress. Recently, rapid industrialization, resulting in air pollution and ozone layer destruction, has led to an increase in the amount of UV. This increases the generation of powerful oxidizing substances such as ROS, which can destroy connective tissue such as collagen, inhibit cell membrane function, promote DNA mutation, alter protein function, and alter intracellular signaling molecules. This can lead to wrinkles, pigmentation, and other skin aging effects. To prevent this damage, cosmetics or skin protectants containing various skin-protecting components, such as retinol, retinoids, vitamin C, flavonoids, tocopherols, and coenzyme Q10, are being developed.

Ultraviolet (UV) rays from sunlight, in particular, are a well-known cause of aging. Prolonged exposure to UV rays causes the stratum corneum to thicken, and collagen and elastin, key components of skin, to degenerate, leading to a loss of elasticity. Collagen and elastin are regulated by various factors. The expression of matrix metallo protease, such as collagenase and elastase, degrades collagen and elastin, ultimately resulting in a decrease in collagen content within the skin. Various substances have been developed and used to suppress the decline in collagen and elastin, which contributes to this loss of elasticity. Among these, retinol and retinoic acid have demonstrated elasticity-enhancing effects, and protein fractions derived from Leguminosae seeds have also been used for their elasticity-enhancing effects. However, these retinoids have the disadvantage of causing skin irritation and allergies even when applied in very small doses.

Recently, in order to develop cosmetics that are relatively less toxic and more eco-friendly, physiologically active substances such as whitening, anti-aging, anti-wrinkle, anti-oxidation, and anti-inflammation substances have been explored through extraction processes and fermentation technologies using natural substances, and these substances are then used as cosmetic materials.

In the present disclosure, while seeking an extraction method capable of extracting large quantities of effective components such as polyphenols and flavonoids, natural raw materials were ground and powdered, and experiments were conducted applying a high-temperature and pressurization extraction process to the powdered raw materials. As a result, it was confirmed that the extract extracted through the high-temperature and pressurization process had significantly increased polyphenol and flavonoid contents compared to previously known extraction processes, thereby completing the present disclosure.

### PRIOR ART DOCUMENTS

### Patent Documents

(Patent Document 0001) Korean Patent Publication No. 10-2017-0122684

### SUMMARY

Therefore, an object of the present disclosure is to provide an extraction method capable of effectively increasing the extraction yield of polyphenols and flavonoids.

Another object of the present disclosure is to provide an extract with increased polyphenol and flavonoid content produced by the extraction method.

Another object of the present disclosure is to provide a cosmetic composition including the extract as an effective component.

To achieve the above-described object of the present disclosure, [0012] the present disclosure provides a method for preparing an extract with increased polyphenol and flavonoid content, comprising the steps of: a) washing and drying a raw material and then grinding the raw material to a size of 0.3 to 0.7 mm; b) extracting an extract by applying high-temperature steam under a pressure of 8 to 10 bar; and c) filtering and then concentrating the extract.

In one embodiment of the present disclosure, the raw material may be one selected from the group consisting of Camellia japonica L., Quercus acuta Thunb., Cinnamomum japonicum Siebold, and edible mushrooms.

In one embodiment of the present disclosure, the polyphenol may be at least one selected from the group consisting of 4-hydroxybenzoic acid, coumaric acid, rutin, naringenin, gallic acid, protocatechuic acid, chlorogenic acid, catechin, and tannic acid.

Furthermore, the present disclosure provides an extract having an increased content of polyphenols and flavonoids produced by the method.

Furthermore, the present disclosure provides a cosmetic composition including the extract of the present disclosure as an effective component.

The high-temperature and pressurization steam extraction process of the present disclosure can significantly increase the extractive contents of protocatechuic acid and chlorogenic acid, as well as gallic acid, which is a powerful antioxidant as an effective component. In addition, the extractive contents of catechin and tannic acid, which are effective in inhibiting wrinkle formation, can also be effectively increased. The extract extracted through this extraction method contains a high content of polyphenol and flavonoid components, and therefore, the composition of the present disclosure containing the extract as an effective component can be usefully used as a variety of functional cosmetic compositions such as antioxidant, wrinkle improvement, and whitening component.

### BRIEF DESCRIPTION OF THE DRAWING

FIG. 1 is an extraction process diagram briefly illustrating the high-temperature and pressurization steam extraction process of the present disclosure.

### DETAILED DESCRIPTION OF THE EMBODIMENTS

The present disclosure is characterized by providing a method for preparing an extract with increased polyphenol and flavonoid content, comprising the steps of: a) washing and drying a raw material and then grinding the raw material to a size of 0.3 to 0.7 mm; b) extracting an extract by applying high-temperature steam under a pressure of 8 to 10 bar; and c) filtering and then concentrating the extract.

In step a), the raw material may be a variety of medicinal or edible plants to be extracted.

In one specific embodiment of the present disclosure, the raw material may be Camellia japonica L., Quercus acuta Thunb., Cinnamomum japonicum Siebold, or edible mushrooms, but the type thereof is not particularly limited.

The ‘Camellia japonica L.’ described in this specification belongs to the Theaceae family and is an evergreen broad-leaved small tree that grows in Gochang, Jeollabuk-do; Haenam, Wando, Gangjin, Yeosu, and Gwangyang, Jeollanam-do; Geoje, the southern coast, Gyeongsangnam-do; and Jeju Island. The Camellia japonica L. grows to about 15 m in height and about 50 cm in diameter. The leaves have a dark green, glossy upper surface and a yellowish-green underside. The leaves are oval and opposite, 5 to 12 cm long and 3 to 7 cm wide, and have wavy, finely serrated edges.

The ‘Quercus acuta Thunb.’ described in this specification belongs to the Fagaceae family and is also called Gasaenamu, Garangnip (Jeolla-do), Gasinang, Bukgasi-nang, Honggasinang, and Berenang (Jeju-do). The Quercus acuta Thunb. grows on sunny mountain slopes and valleys, reaching 20 meters in height and 60 cm in diameter. The trunk thereof grows straight, with numerous branches and lush leaves, forming a magnificent tree. The ‘Quercus acuta Thunb.’ is a temperate evergreen broad-leaved tree and is a common species in temperate regions of Korea and Japan. ‘Quercus acuta Thunb.’ was a key tree in the temperate forests of southern islands, including Jeju Island, and the southern coastal mountains. The name ‘Quercus acuta Thunb.’ derives from the red color of the wood thereof. The tannins in Quercus acuta Thunb.'s fruit have astringent properties and are known to have antidiarrheal properties, and are known to be effective in treating dysentery, colitis, diarrhea, milk fever, gingivitis, and stomatitis.

The “Cinnamomum japonicum Siebold” described in this specification is an evergreen broadleaf tree belonging to the Lauraceae family, with a pungent and sweet flavor. In Oriental medicine, the bark and fruit of the tree are used as a medicinal herb called Cheonchukgye. These bark and fruit contain components such as phellandrene, eugenol, and methyleugenal, which are reported to be effective in enhancing digestive power and treating vomiting, dysentery, neuralgia, or the like. Furthermore, the leaves of the Cinnamomum japonicum Siebold have a strong fragrance and have long been used as a tea substitute.

Currently, hot water extraction and organic solvent extraction are the main extraction methods for extracting effective components from these plants.

In the present disclosure, the polyphenol and flavonoid contents of extracts of Camellia japonica L., Quercus acuta Thunb., and Cinnamomum japonicum Siebold, respectively, using different extraction methods, were measured. As a result, it was confirmed for the first time that the polyphenol and flavonoid contents were significantly increased in extracts extracted using the high-temperature and pressurization steam extraction method of the present disclosure compared to existing extraction methods.

The polyphenols include 4-hydroxybenzoic acid, coumaric acid, rutin, naringenin, gallic acid, protocatechuic acid, chlorogenic acid, catechin, and tannic acid.

Furthermore, the present disclosure provides an extract with increased polyphenol and flavonoid contents produced by the method.

The extract contains a large amount of protocatechuic acid and chlorogenic acid, as well as gallic acid, a powerful antioxidant, as effective components. The extract also contains a large amount of catechin and tannic acid, which are effective in inhibiting wrinkle formation.

Furthermore, the present disclosure provides a cosmetic composition including the extract as an effective component.

The cosmetic composition of the present disclosure can be used for various functional purposes, such as antioxidant, wrinkle improvement, and whitening.

In one specific embodiment of the present disclosure, the extract may be included in a cosmetic composition at a concentration of 0.0001 to 2000 μg/ml.

Products to which the cosmetic composition of the present disclosure can be added include, for example, cosmetics such as astringent toners, emollient toners, nourishing toners, various creams, essences, packs, foundations, and cleansing products, facial cleansers, soaps, treatments, and beauty solutions.

Specific formulations of the cosmetic composition of the present disclosure include one formulation selected from the group consisting of emollient toners, gels, water-soluble liquids, milk lotions, nourishing creams, massage creams, essences, oil-in-water emulsions, water-in-oil emulsions, anhydrous face products, anhydrous solid products, oil dispersions in aqueous phases using spherules, ionic lipid vesicles, nonionic lipid vesicles, ointments, cleansing foams, cleansing water, packs, body oils, oil-in-water makeup bases, water-in-oil makeup bases, foundations, skin covers, lipsticks, lip glosses, face powders, two-way cakes, eye shadows, mascara, cheek colors, and eyebrow pencils.

According to a preferred embodiment of the present disclosure, the content of the effective component (extract extracted by high-temperature and pressurization steam extraction) of the present disclosure is 0.00001-40 wt %, preferably 0.0005-40 wt %, and more preferably 0.0005-20 wt %, based on the total weight of the composition.

Meanwhile, the cosmetic composition according to the present disclosure can be formulated by stabilizing the effective component (extract extracted by high-temperature and pressurization steam extraction) by containing the effective component within nanoliposomes. By containing the effective component within nanoliposomes, the effective component is stabilized, thereby resolving problems such as precipitation, discoloration, and odor change during formulation. In addition, the solubility and percutaneous absorption rate of the component can be increased, thereby maximizing the efficacy expected from the extract.

In the present disclosure, nanoliposomes have the form of conventional liposomes and mean liposomes with an average particle diameter of 10 to 500 nm. According to a preferred embodiment of the present disclosure, the average particle diameter of the nanoliposomes is 50 to 300 μm, and more preferably 100 to 200 nm. When the average particle diameter of the nanoliposomes exceeds 500 nm, the technical effects to be achieved in the present disclosure, such as improved skin penetration and improved formulation stability, are very weak.

According to the present disclosure, nanoliposomes used to stabilize the effective component (an extract extracted by high-temperature and pressurization steam extraction) can be manufactured using a mixture including a polyol, an oily component, a surfactant, a phospholipid, a fatty acid, and water.

The polyol used in the nanoliposome of the present disclosure is not particularly limited, and is preferably at least one selected from the group consisting of propylene glycol, dipropyl glycol, 1,3-butylene glycol, glycerin, methylpropanediol, isopropylene glycol, pentylene glycol, erythritol, xylitol, sorbitol, and mixtures thereof. The amount used is 10 to 80 wt %, preferably 30 to 70 wt %, based on the total weight of the nanoliposome.

The oil component used in the preparation of the nanoliposome of the present disclosure may be various oils known in the art, and preferably includes hydrocarbon oils such as hexadecane and paraffin oil, synthetic ester oils, silicone oils such as dimethicone and cyclomethicone, animal and vegetable oils such as sunflower oil, corn oil, soybean oil, avocado oil, sesame oil, and fish oil, ethoxylated alkyl ether oils, propoxylated alkyl ether oils, sphingonoid lipids such as phytosphingosine, sphingosine, and sphinganine, cerebroside cholesterol, sitosterol cholesteryl sulfate, sitosteryl sulfate, C₁₀₋₄₀ fatty alcohol and mixtures thereof. The amount used may be 1.0 to 30.0 wt %, and preferably 3.0 to 20.0 wt %, based on the total weight of the nanoliposome.

Any surfactant known in the art can be used in the preparation of the nanoliposomes of the present disclosure. For example, anionic surfactants, cationic surfactants, amphoteric surfactants, and nonionic surfactants can be used. Anionic surfactants and nonionic surfactants are preferred. Specific examples of anionic surfactants include alkylacyl glutamates, alkyl phosphates, alkyl lactylates, dialkyl phosphates, and trialkyl phosphates. Specific examples of nonionic surfactants include alkoxylated alkyl ethers, alkoxylated alkyl esters, alkyl polyglycosides, polyglyceryl esters, and sugar esters. Particularly preferred surfactants are polysorbates, which belong to the nonionic surfactants. The amount used may be 0.1 to 10 wt %, and preferably 0.5 to 5.0 wt %, based on the total weight of the nanoliposome.

Another component used in the preparation of the nanoliposome of the present disclosure, the phospholipid, is an amphiphilic lipid and includes natural phospholipids (for example, egg yolk lecithin or soy lecithin, sphingomyelin) and synthetic phospholipids (for example, dipalmitoylphosphatidylcholine or hydrogenated lecithin), and is preferably lecithin. In particular, natural unsaturated lecithin or saturated lecithin extracted from soybean or egg yolk is preferred. Typically, natural lecithin contains 23 to 95% phosphatidylcholine and 20% or less phosphatidylethanolamine. In the preparation of the nanoliposomes of the present disclosure, the amount of phospholipid used is 0.5 to 20.0 wt %, and preferably 2.0 to 8.0 wt %, based on the total weight of the nanoliposomes.

The fatty acid used in the preparation of the nanoliposomes of the present disclosure is a higher fatty acid, preferably a saturated or unsaturated fatty acid with a C₁₂₋₂₂ alkyl chain, and includes lauric acid, myristic acid, palmitic acid, stearic acid, oleic acid, and linoleic acid. The amount used may be 0.05 to 3.0 wt %, and preferably 0.1 to 1.0 wt %, based on the total weight of the nanoliposomes.

The water used in the preparation of the nanoliposomes of the present disclosure is generally deionized distilled water, and the amount used may be 5.0 to 40 wt %, based on the total weight of the nanoliposomes.

Nanoliposomes can be prepared through various methods known in the art. However, most preferably, the mixture containing the components is applied to a high-pressure homogenizer. The preparation of nanoliposomes using a high-pressure homogenizer can be performed under various conditions (for example, pressure, number of passes, or the like) according to the desired particle size. Preferably, the nanoliposomes are prepared by passing the mixture through the high-pressure homogenizer 1 to 5 times under a pressure of 600 to 1200 bar.

The cosmetic composition of the present disclosure can be applied alone or in combination, or in combination with other cosmetic compositions. Furthermore, the cosmetic composition of the present disclosure can be used according to conventional methods, and the frequency of application can vary according to the user's skin condition or preference.

The present disclosure will now be described in more detail through examples. These examples are intended to further specifically illustrate the present disclosure, and the scope of the present disclosure is not limited to these examples.

### Example 1

### Preparation of Extracts of Camellia japonica L., Quercus acuta Thunb., And Cinnamomum japonicum Siebold Through High-Temperature and Pressurization Extraction Process

Leaves of Camellia japonica L. used in this experiment were collected from the Wando Arboretum; Quercus acuta Thunb. branches were collected from the Jeollanam-do Forest Resources Research Institute; and the leaves and branches of Cinnamomum japonicum Siebold were collected from the Jeollanam-do Forest Resources Research Institute.

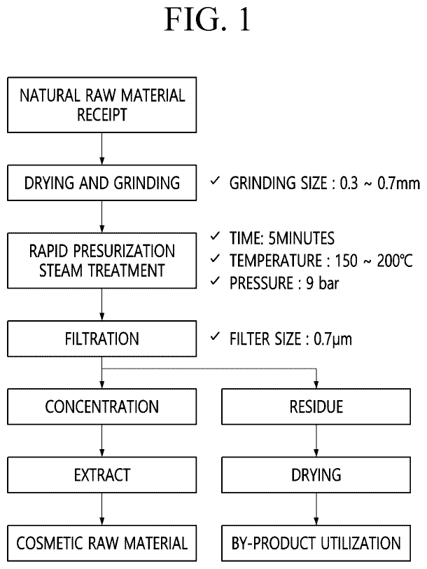

Each of the natural raw materials prepared above was washed and dried, ground to a size of 0.3-0.7 mm, and then used as a powder. Each of the ground natural raw materials was pressed into a high-temperature and pressurization device and extracted in water at 150-200° C. for 5 minutes. The extracted extracts were filtered through a 0.71 μm filter to remove microorganisms and foreign substances. The filtered filtrate was processed using a rotary vacuum evaporator to produce a concentration of 50-1,000 μg/mL for use. The process for preparing extracts of Camellia japonica L., Quercus acuta Thunb. and Cinnamomum japonicum Siebold using the high-temperature and pressurization extraction process of the present disclosure is detailed in FIG. 1.

The extraction yield was calculated using Equation 1 below.

$$Yield = ( Weight of sample after drying ( g ) / Weight of sample before extraction ( g ) ) \times 100 \qquad [ Equation 1 ]$$

TABLE 1
Yield of extracts obtained through the high-temperature and
pressurization extraction process of the present disclosure

| | Camellia | Quercus acuta | Cinnamomum |
|---|---|---|---|
| Extracts | japonica L. | Thunb. | japonicum Siebold |
| Yield (%) | 22.90 | 22.37 | 12.90 |
| | (22.90 g) | (22.37 g) | (12.90 g) |

### Comparative Example 1

### Preparation of Extracts of Camellia japonica L., Quercus acuta Thunb., and Cinnamomum japonicum Siebold Through an Existing Extraction Process

Each prepared natural raw material was washed and dried, ground to a 0.3-0.7 mm particle size, and then used as a powder. After steeping 100 g of leaf powder in 1,000 ml of distilled water, extraction was performed using an autoclave at 100° C. for 10 minutes. The extracted extract was filtered through filter paper (Whatman No. 2) and concentrated using a rotary vacuum evaporator for use as a sample.

The extraction yield was calculated using Equation 1.

TABLE 2
Yields of extracts obtained using an existing extraction process

| | Camellia | Quercus acuta | Cinnamomum |
|---|---|---|---|
| Extracts | japonica L. | Thunb. | japonicum Siebold |
| Yield (%) | 15.27 | 10.96 | 5.00 |
| | (15.27 g) | (10.96 g) | (5.00 g) |

### Experimental Example 1

### Evaluation of Antioxidant Activity of Extracts

To evaluate the antioxidant activity of the extracts of the Camellia japonica L., Quercus acuta Thunb., and Cinnamomum japonicum Siebold prepared in <Example 1> and <Comparative Example 1>, DPPH free radical scavenging activity and ABTS radical scavenging activity were examined.

### <1-1> DPPH Free Radical Scavenging Activity

DPPH free radical scavenging activity is a widely used method for measuring the antioxidant activity of natural extracts. In this experiment, the radical scavenging activity of the samples was measured using a modification of Blois' method, one of the antioxidant activity measurement methods. Specifically, 800 μL of 0.25 mM DPPH (2,2-diphenyl-1-picrylhydrazyl, Sigma, USA) dissolved in methanol and 200 μL of each sample (50-1,000 □g/mL) were placed in an EP tube, vortexed, and reacted in a darkroom for 15 minutes. Afterwards, the absorbance was measured at 517 nm using an HT multi-detection microplate reader. The DPPH scavenging activity (IC₅₀) of the extracts was expressed as the concentration required to reduce the absorbance by 50% of the control group using only the solvent.

$$Antioxidant activity ( \% ) = \frac{absorbance of control - absorbance of sample}{absorbance of control} \times 100$$

As a result, the IC₅₀ value of the Camellia japonica L. extract extracted through the existing extraction method was 443.17 ug/mL, whereas the IC₅₀ value of the Camellia japonica L. extract extracted through the high-temperature and pressurization extraction method of the present disclosure was 262.99 ug/mL, illustrating increase in an antioxidant activity of approximately 40.7%.

In addition, the IC₅₀ value of the Quercus acuta Thunb. extract extracted through the existing extraction method was 340.70 ug/mL, whereas the IC₅₀ value of the Quercus acuta Thunb. extract extracted through the high-temperature and pressurization extraction method of the present disclosure was 250.34 ug/mL, illustrating increase in an antioxidant activity of approximately 26.5%.

Furthermore, while the extract of the Cinnamomum japonicum Siebold extracted through the existing extraction method had an IC₅₀ value of 346.81 μg/mL, the extract of the Cinnamomum japonicum Siebold extracted through the high-temperature and pressurization extraction method of the present disclosure had an IC₅₀ value of 163.82 ug/mL, illustrating increase in an antioxidant activity of a 52.7%.

### <1-2> ABTS Radical Scavenging Activity

ABTS radical scavenging activity has the advantage of being able to measure the radical scavenging activity of both hydrophilic and hydrophobic samples, offering a wide range of applications. In this experiment, the method of Jeong et al. was modified to measure the radical scavenging activity. Specifically, a 7 mM ABTS solution dissolved in distilled water and 2.45 mM potassium persulfate were mixed in a 1:1 ratio and left in the dark for 16 hours. Radical stock solution was diluted with PBS (pH 7.4) to an absorbance value of 0.70±0.02. 200 μL of samples prepared at various concentrations (50-1,000 ng/mL) were added to 1,000 uL of the diluted solution and reacted in a darkroom for 15 minutes. Afterwards, the absorbance was measured at 517 nm using an HT multi-detection microplate reader (Synergy HT, BIO-TEX, Winooski, VT, USA). The scavenging activity (IC₅₀) of the extract for ABTS was expressed as the concentration required to reduce the absorbance of the control group using only solvent by 50%.

$$Antioxidant activity ( \% ) = \frac{absorbance of control - absorbance of sample}{absorbance of control} \times 100$$

As a result, the IC₅₀ value of the extract of the Camellia japonica L. extracted through the existing extraction method was 463.03 ug/mL, whereas the extract of the Camellia japonica L. extracted through the high-temperature and pressurization extraction method of the present disclosure illustrated an IC₅₀ value of 260.09 ug/mL, illustrating increase in an antioxidant activity of approximately 43.8%.

In addition, the IC₅₀ value of the extract of the Quercus acuta Thunb. extracted through the existing extraction method was 199.31 ug/mL, whereas the extract of the Quercus acuta Thunb. extracted through the high-temperature and pressurization extraction method of the present disclosure illustrated an IC₅₀ value of 117.54 □g/mL, illustrating increase in an antioxidant activity of approximately 41.0%.

In addition, while the IC₅₀ value of the extract of the Cinnamomum japonicum Siebold extracted through the existing extraction method was 57.69 ug/mL, the IC₅₀ value of the extract of the Cinnamomum japonicum Siebold extracted through the high-temperature and pressurization extraction method of the present disclosure was 41.07 ug/mL, illustrating increase in an antioxidant activity of 28.8%.

TABLE 3
Antioxidant activity of the extract

| | | DPPH IC₅₀ | ABTS IC₅₀ |
|---|---|---|---|
| Sample | | (ug/mL) | (μg/mL) |
| Camellia | Existing extraction | 443.17 ± 5.99 | 463.03 ± 4.54 |
| japonica L. | High-temperature | 262.99 ± 1.83 | 260.09 ± 0.6 |
| | and pressurization | | |
| | extraction | | |
| Quercus | Existing extraction | 340.70 ± 2.57 | 199.31 ± 0.81 |
| acuta | High-temperature | 250.34 ± 2.64 | 117.54 5.72 |
| Thunb. | and pressurization | | |
| | extraction | | |
| Cinnamomum | Existing extraction | 346.81 ± 8.68 | 57.69 ± 8.12 |
| japonicum | High-temperature | 163.82 ± 19.63 | 41.07 ± 4.62 |
| Siebold | and pressurization | | |
| | extraction | | |

### Experimental Example 2

### Total Polyphenol and Total Flavonoid Contents of Extracts

### <2-1> Analysis of Total Polyphenol Content

Polyphenols are aromatic alcohol compounds found in plants, each molecule containing two or more phenol groups. The polyphenols have a variety of structures and molecular weights, and their hydroxyl groups facilitate binding to various compounds, including water-soluble proteins. Polyphenols have various physiological functions, such as anticancer, anti-inflammatory, and antioxidant properties, and their functions vary according to the content and components. In this experiment, the total polyphenol content of the extracts of Camellia japonica L., Quercus acuta Thunb., and Cinnamomum japonicum Siebold was measured according to the extraction method.

Total polyphenol content analysis was measured using the Folin-Ciocalteu method (Ainsworth, E. A., & Gillespie, K. M., Nat. Protoc., 2(4), 875-877(2007).). Briefly, the extract concentration was prepared at 500 μg/mL, and 500 μL of 0.2 M Folin-Ciocalteu's phenol reagent and then 500 μL of 2% sodium carbonate aqueous solution (w/v) were sequentially mixed with 500 L of the prepared sample, and the mixture was reacted in a dark room at room temperature for 30 minutes. Afterwards, the absorbance was measured at 750 nm using an HT multi-detection microplate reader. A calibration curve was created using gallic acid diluted at various concentrations as a standard substance, and then the mg/g gallic acid (GAE) equivalent was converted using the calibration curve.

As a result, the total polyphenol contents of the extract of the Camellia japonica L., the extract of the Quercus acuta Thunb., and the extract of the Cinnamomum japonicum Siebold extracted through the high-temperature and pressurization extraction method of the present disclosure illustrated increases of approximately 87.1%, approximately 32.3%, and approximately 18.5%, respectively, compared to the existing extraction method (see Table 4).

### <2-2> Analysis of Total Flavonoid Content

Flavonoids, which exist in various forms in plants, belong to the polyphenol group and possess significant anti-inflammatory, anti-cancer, anti-obesity, and antioxidant effects. In this experiment, the total flavonoid content of extracts from Camellia japonica L., Quercus acuta Thunb., and Cinnamomum japonicum Siebold, according to the extraction method, was measured.

To analyze total flavonoid content, after the extract was prepared at a concentration of 1,000 ng/mL, 500 μL of the prepared sample was sequentially mixed with 1.5 mL of methanol, 100 μL of 1 M potassium acetate, and 1.4 mL of the distilled water. After reaction at room temperature for 40 minutes, the absorbance was measured at 415 nm using an HT multi-detection microplate reader. A calibration curve was developed using quercetin diluted at various concentrations as a standard material, and then the mg/g quercetin (QUE) equivalent was then calculated.

As a result, the total flavonoid contents of the extract of the Camellia japonica L., the extract of the Quercus acuta Thunb., and the extract of the Cinnamomum japonicum Siebold extracted through the high-temperature and pressurization extraction method of the present disclosure increased by approximately 20.6%, 157.3%, and 880.7%, respectively, compared to the existing extraction method (see Table 4).

TABLE 4
Total polyphenol and Total flavonoid contents of the extracts

| | | TPC | TFC |
|---|---|---|---|
| Sample | | (GAE mg/g) | (QUE mg/g) |
| Camellia | Existing extraction | 89.75 ± 1.38 | 32.76 ± 1.42 |
| japonica L. | High-temperature | 167.88 ± 1.57 | 39.50 ± 0.39 |
| | and pressurization | | |
| | extraction | | |
| Quercus | Existing extraction | 120.93 ± 0.76 | 22.11 ± 0.30 |
| acuta | High-temperature | 159.99 ± 0.97 | 56.87 ± 0.60 |
| Thunb. | and pressurization | | |
| | extraction | | |
| Cinnamomum | Existing extraction | 167.53 ± 1.00 | 4.30 ± 0.64 |
| japonicum | High-temperature | 198.56 ± 4.38 | 42.17 ± 4.04 |
| Siebold | and pressurization | | |
| | extraction | | |

### Experimental Example 3

### Component Analysis Using LC-MS/MS Analysis of Extracts

To quantitatively analyze the effective components contained in the extract of the Camellia japonica L., the extract of Quercus acuta Thunb., and the extract of Cinnamomum japonicum Siebold prepared in <Example 1> and <Comparative Example 1>, HPLC-MS/MS (AB SCIEX 4000 Q Trap LC/MS/MS System, Shimadzu LC 20A System) was used to analyze nine polyphenols (4-hydroxybenzoic acid); coumaric acid; rutin; Naringenin; gallic acid; protocatechuic acid; chlorogenic acid; catechin; tannic acid). A sample with an injection volume of 10 L was analyzed using an autosampler (15° C.) under column oven (40° C.) conditions using a C18 column (Gemini 3 um, C18 110A 50 mm×2.0 mm). Analysis was performed in negative and positive ion modes using Turbo Ion Spray, and water (0.1% formic acid) (A) and acetonitrile (0.1% formic acid) (B) were used as mobile phases. The anion mode was analyzed under isocratic conditions based on the mobile phase (B) as follows: 0-0.5 min: B (20-20%), 0.5-2 min: B (20-80%), 2-2.5 min: B (80-80%), 2.5-2.6 min: B (80-20%), 2.6-6 min: B (20-20%), and 0-3 min: B (30-30%), and the positive ion mode was analyzed as follows: 0-0.2 min: B (30-60%), 0.2-2 min: B (60-60%), 2-2.1 min: B (60-30%), and 2.1-4 min: B (30-30%).

The results are detailed in Table 5.

As a result of analyzing polyphenols using LC-MS/MS, it was found that the content of hydroxybenzoic acid increased by 380%, coumaric acid by 217.7%, rutin by 573.2%, naringenin by 139.3%, gallic acid by 787.4%, protocatechuic acid by 814.1%, catechin by 280.2%, and tannic acid by 9,543% in the extract of the Camellia japonica L. extracted through the high-temperature and pressurization extraction method of the present disclosure compared to the existing extraction method.

Compared to the existing extraction method, in the extract of the Quercus acuta Thunb. extracted through the high-temperature and pressurization extraction method of the present disclosure, the content of hydroxybenzoic acid increased by 149.6%, coumaric acid by 3,720%, naringenin by 453.8%, gallic acid by 1,710.2%, protocatechuic acid by 48,483.1%, and catechin by 63.9% and tannic acid by 508%, respectively.

Compared to the existing extraction method, in the extract of the Cinnamomum japonicum Siebold. extracted through the high-temperature and pressurization extraction method of the present disclosure, the content of hydroxybenzoic acid increased by 687.7%, rutin by 590.6%, naringenin by 131.6%, gallic acid by 12,214.5%, and protocatechuic acid by 363.3%. In particular, chlorogenic acid, catechin, and tannic acid were not detected in the extract extracted using the existing extraction method, but were detected at 2,362.70 μg/g, 4.16 μg/g, and 2,800 μg/g, respectively, in the extract of the Cinnamomum japonicum Siebold extracted through the high-temperature and pressurization extraction method of the present disclosure.

TABLE 5
LC-MS/MS analysis results of the extract

| | | Concentration (μg/g) | | | | | |
|---|---|---|---|---|---|---|---|
| | | | | | | Cinnamomum japonicum | |
| | | Camellia japonica L. | | Quercus acuta Thunb. | | Siebold | |
| | | | High- | | High- | | High- |
| | | | temperature | | temperature | | temperature |
| | | | and | | and | | and |
| | Standard | Existing | pressurization | Existing | pressurization | Existing | pressurization |
| No. | material | extraction | extraction | extraction | extraction | extraction | extraction |
| 1 | 4-hydroxybenzoic | 15.05 | 72.22 | 3.87 | 9.66 | 45.60 | 359 |
| | acid | | | | | | |
| 2 | Coumaric acid | 9.17 | 29.16 | 0.77 | 29.40 | — | — |
| 3 | Rutin | 682.93 | 4.597.17 | — | — | 41.40 | 286 |
| 4 | Naringein | 0.84 | 2.01 | 0.52 | 2.88 | 9.37 | 21.70 |
| 5 | Gallic acid | 220.01 | 1,953.26 | 7.57 | 137 | 11.70 | 1,440 |
| 6 | Protocatechuic | 101.36 | 926.27 | 8.31 | 4,030 | 95.40 | 442 |
| | acid | | | | | | |
| 7 | chlorogenic | — | — | — | 2.87 | — | 2,362.70 |
| | acid | | | | | | |
| 8 | Catechin | 1,629.42 | 6,195.48 | 7,320 | 12,000 | — | 4.16 |
| 9 | Tannic acid | 1.15 | 111 | 250 | 1520 | — | 2,800 |

For reference, gallic acid is a powerful antioxidant, and protocatechuic acid is a type of phenolic acid and a major metabolite of the antioxidant polyol found in green tea, known to protect the skin from oxidative stress caused by free radicals. Chlorogenic acid is a polyphenol found in nature and is one of the powerful antioxidants that relieves oxidative stress in cells and prevents the occurrence of various symptoms and diseases caused by oxidative stress. Catechin is known to affect human skin cells involved in the cause of aging, inhibiting wrinkle formation and providing moisture and elasticity to the skin. Tannic acid has been reported to inhibit elastase activity in a concentration-dependent manner, so it is known to be a substance that inhibits wrinkle formation.

Therefore, compared to existing extraction methods, the high-temperature and pressurization extraction method of the present disclosure significantly increases the extraction content of protocatechuic acid and chlorogenic acid, as well as gallic acid, a powerful antioxidant. Furthermore, the content of catechin and tannic acid, which are effective in inhibiting wrinkle formation, can also be significantly increased. Therefore, the extract extracted using the high-temperature and pressurization extraction method of the present disclosure can be useful as a functional material for improving wrinkles, moisturizing the skin, and whitening.

### Formulation Example 1: Emollient Toners (Skin Lotion)

As illustrated in the table below, an emollient toners was prepared according to an existing method.

TABLE 6
Formulation Example of an Emollient
Toners of the Present Disclosure

| Mixing components | Content (weight %) |
|---|---|
| extracts of <Example 1> | 1~5 |
| 1,3-butylene glycol | 6.0 |
| glycerin | 4.0 |
| DL-panthenol | 0.3 |
| allantoin | 0.1 |
| polysorbate 20 | 0.5 |
| ethanol | 15.0 |
| benzophenone-9 | 0.05 |
| incense, preservatives | very small amount |
| purified water | to 100 |

### Formulation Example 2: Nourishing Lotion (Milk Lotion)

As illustrated in the table below, a nourishing lotion was prepared according to an existing method.

TABLE 7
Formulation Example of a Nourishing
Lotion of the present disclosure

| Mixing components | Content (weight %) |
|---|---|
| extracts of <Example 1> | 1~5 |
| glyceryl stearate SE | 1.5 |
| Cetearyl alcohol | 1.5 |
| Lanolin | 1.5 |
| polysorbate 60 | 1.3 |
| sorbitan stearate | 0.5 |
| hydrogenated vegetable oils | 4.0 |
| mineral oil | 5.0 |
| trioctanoin | 2.0 |
| dimethicone | 0.8 |
| tocopherol acetate | 0.5 |
| carboxyvinyl polymer | 0.12 |
| glycerin | 5.0 |
| 1,3-butylene glycol | 3.0 |
| sodium hyaluronate | 5.0 |
| Triethanolamine | 0.12 |
| uniside-U 13 | 0.02 |
| incense | very small amount |
| distilled water | to 100 |

### Formulation Example 3: Nourishing Cream

A nourishing cream was prepared according to an existing method, as illustrated in the table below.

TABLE 8
Formulation Example of a Nourishing
Cream of the Present Disclosure

| Mixing components | Content (weight %) |
|---|---|
| extracts of <Example 1> | 1~5 |
| lipophilic monostearic acid glycerin | 1.5 |
| cetearyl alcohol | 1.5 |
| stearic acid | 1.0 |
| polysorbate 60 | 1.5 |
| sorbitan stearate | 0.6 |
| isostearyl isostearate | 5.0 |
| squalane | 5.0 |
| mineral oil | 35.0 |
| dimethicone | 0.5 |
| hydroxyethylcellulose | 0.12 |
| glycerin | 6.0 |
| triethanolamine | 0.7 |
| uniside-U13 | 0.02 |
| incense | very small amount |
| distilled water | to 100 |

### Formulation Example 4: Massage Cream

Massage cream was prepared according to existing methods, as illustrated in the table below.

TABLE 9
Formulation Example of a Massage
Cream of the Present Disclosure

| Mixing components | Content (weight %) |
|---|---|
| extracts of <Example 1> | 1~5 |
| propylene glycol | 6 |
| glycerin | 4.0 |
| triethanolamine | 0.5 |
| beeswax | 2.0 |
| tocopheryl acetate | 0.1 |
| polysorbate 60 | 3.0 |
| sorbitan sesquioleate | 2.5 |
| cetearyl alcohol | 2.0 |
| liquid paraffin | 30.0 |
| carboxyvinyl polymer | 0.5 |
| incense | very small amount |
| distilled water | to 100 |

### Formulation Example 5: Pack

A pack was prepared according to an existing method, as illustrated in the table below.

TABLE 10
Formulation Example of a Pack of the Present Disclosure

| Mixing components | Content (weight %) |
|---|---|
| extracts of <Example 1> | 1~5 |
| glycerin | 10.0 |
| betaine | 5.0 |
| REG 1500 | 2.0 |
| allantoin | 0.1 |
| DL-panthenol | 0.3 |
| EDTA-2Na | 0.02 |
| benzophenone-9 | 0.04 |
| hydroxyethyl cellulose | 0.1 |
| sodium hyaluronate | 80. |
| carboxyvinyl polymer | 0.2 |
| triethanolamine | 0.18 |
| octyldodecanol | 0.3 |
| octyldodeces-16 | 0.4 |
| ethanol | 6.0 |
| uniside-13 | 0.01 |
| incense | very small amount |
| distilled water | To 100 |

The present disclosure has been described with a focus on preferred embodiments thereof. Those skilled in the art will appreciate that the present disclosure can be implemented in modified forms without departing from the essential characteristics thereof. Therefore, the disclosed embodiments should be considered illustrative rather than limiting. The scope of the present disclosure is defined by the claims, not the foregoing description, and all differences within the scope equivalent thereto should be construed as encompassed by the present disclosure.

What is claimed is:

1. A method for preparing an extract with increased polyphenol and flavonoid content, comprising:
a) washing and drying a raw material and then grinding the raw material to a particle size of 0.3 to 0.7 mm;
b) applying high-temperature steam under a pressure of 8 to 10 bar to extract an extract; and
c) filtering and then concentrating the extract.

2. The method for preparing an extract with increased polyphenol and flavonoid content of claim 1, characterized in that the raw material is one selected from the group consisting of Camellia japonica L., Quercus acuta Thunb., Cinnamomum japonicum Siebold, and edible mushrooms.

3. The method for preparing an extract with increased polyphenol and flavonoid content of claim 1, characterized in that the polyphenol is one or more selected from the group consisting of 4-hydroxybenzoic acid, coumaric acid, rutin, naringenin, gallic acid, protocatechuic acid, chlorogenic acid, catechin, and tannic acid.

4. An extract having increased content of polyphenols and flavonoids, produced by the method of the claim 1.

5. A cosmetic composition comprising the extract of claim 4 as an active ingredient.

6. The cosmetic composition according to the claim 5, wherein the extract is included in the cosmetic composition at a concentration of 0.0001 to 2000 μg/ml.

7. The cosmetic composition according to the claim 5, wherein formulations of the cosmetic composition include one formulation selected from the group consisting of emollient toners, gels, water-soluble liquids, milk lotions, nourishing creams, massage creams, essences, oil-in-water emulsions, water-in-oil emulsions, anhydrous face products, anhydrous solid products, oil dispersions in aqueous phases using spherules, ionic lipid vesicles, nonionic lipid vesicles, ointments, cleansing foams, cleansing water, packs, body oils, oil-in-water makeup bases, water-in-oil makeup bases, foundations, skin covers, lipsticks, lip glosses, face powders, two-way cakes, eye shadows, mascara, cheek colors, and eyebrow pencils.

8. The cosmetic composition according to the claim 5, wherein the content of the effective component is 0.00001-40 wt %, based on the total weight of the composition.

9. The cosmetic composition according to the claim 5, wherein the content of the effective component is 0.0005-40 wt %, based on the total weight of the composition.

10. The cosmetic composition according to the claim 5, wherein the content of the effective component is 0.0005-20 wt %, based on the total weight of the composition.

11. The cosmetic composition according to the claim 5, wherein the cosmetic composition is formulated by stabilizing the effective component by containing the effective component within nanoliposomes.

12. The cosmetic composition according to the claim 11, wherein the nanoliposomes have an average particle diameter of 10 to 500 nm.

13. The cosmetic composition according to the claim 11, wherein the nanoliposomes used to stabilize the effective component is manufactured using a mixture including a polyol, an oily component, a surfactant, a phospholipid, a fatty acid, and water.

14. The cosmetic composition according to the claim 13, wherein the polyol used in the nanoliposome is at least one selected from the group consisting of propylene glycol, dipropyl glycol, 1,3-butylene glycol, glycerin, methylpropanediol, isopropylene glycol, pentylene glycol, erythritol, xylitol, sorbitol, and the amount of the polyol is 10 to 80 wt %, based on the total weight of the nanoliposome.

15. The cosmetic composition according to the claim 13, wherein the oil component in the nanoliposome includes hydrocarbon oils of hexadecane and paraffin oil, synthetic ester oils, silicone oils of dimethicone and cyclomethicone, animal and vegetable oils of sunflower oil, corn oil, soybean oil, avocado oil, sesame oil, and fish oil, ethoxylated alkyl ether oils, propoxylated alkyl ether oils, sphingonoid lipids of phytosphingosine, sphingosine, and sphinganine, cerebroside cholesterol, sitosterol cholesteryl sulfate, sitosteryl sulfate, C₁₀₋₄₀ fatty alcohol and mixtures thereof, and the amount of the oil component is 1.0 to 30.0 wt %, based on the total weight of the nanoliposome.

16. The cosmetic composition according to the claim 13, wherein the surfactant in the nanoliposomes is anionic surfactants, cationic surfactants, amphoteric surfactants, and/or nonionic surfactants, and the amount of the surfactant is 0.1 to 10 wt %, based on the total weight of the nanoliposome.

17. The cosmetic composition according to the claim 13, wherein the phospholipid is an amphiphilic lipid and includes natural phospholipids and synthetic phospholipids, and the amount of the phospholipid is 0.5 to 20.0 wt %, based on the total weight of the nanoliposomes.

18. The cosmetic composition according to the claim 13, wherein the fatty acid is a higher fatty acid, and the amount of the fatty acid is 3.0 wt %, based on the total weight of the nanoliposomes.

19. The cosmetic composition according to the claim 13, wherein the water is deionized distilled water, and the amount of the water is 5.0 to 40 wt %, based on the total weight of the nanoliposomes.

20. The cosmetic composition according to the claim 13, wherein the nanoliposomes is prepared by a high-pressure homogenizer, wherein the preparation of nanoliposomes using a high-pressure homogenizer are prepared by passing the mixture through the high-pressure homogenizer 1 to 5 times under a pressure of 600 to 1200 bar.
